(19) United States
(12) Patent Application Publication
(10) Pub. No.: US 2004/0166232 A1
(43) Pub. Date: Aug. 26, 2004

(54) Surface treatment utilizing microwave radiation

(75) Inventors: Jay F. Kunzler, Canandaigua, NY (US); Joseph A. McGee, DeWitt, NY (US); Joseph C. Salamone, Fairport, NY (US); David E. Seelye, North Chile, NY (US)

Correspondence Address: Bausch & Lomb Incorporated, One Bausch & Lomb Place, Rochester, NY, 14604-2701, US

(73) Assignee: Bausch & Lomb Incorporated, Rochester, NY (US)

(21) Appl. No.: 10/740,003

(22) Filed: Dec. 18, 2003

Related U.S. Application Data
(60) Provisional application No. 60/436,229, filed on Dec. 23, 2002.

(51) Int. Cl. A61L 2/00

(52) U.S. Cl. 427/2.14; 427/2.1

(57) ABSTRACT

This invention provides a method of modifying the surface of a medical device, such as an ophthalmic lens. The method involves contacting a surface of the medical device with a solution containing a surface modifying agent; and subjecting the device surface and surface modifying agent to microwave radiation while the surface modifying agent is in contact with the device surface. The device may be constructed of materials such as hydrogel copolymers and silicone materials.

### FIELD OF THE INVENTION

The present invention relates generally to surface modification of polymeric materials such as those materials used in the manufacture of medical devices including ophthalmic lenses such as contact lenses and intraocular lenses, stents, and catheters and intraocular lens inserters. More specifically, the present invention relates to surface modification of polymeric materials using microwave energy to significantly shorten cycle times and to permit use of lower treatment temperatures typically required for applying biocompatible coatings.

### BACKGROUND OF THE INVENTION

It is often desired to improve the surface characteristics of a medical device. For example, in the case of intraocular lenses, the surfaces of the lenses may be rendered more biocompatible, for the purpose of reducing or eliminating silicone oil absorption and lens epithelial cell growth thereon. In the case of intraocular lens inserters, there are surfaces that contact the lens while it is extruded against these surfaces; these surfaces may be modified to become more lubricious so as to lower the coefficient of friction. In the case of contact lenses, the lens surfaces may be made more wettable by tear film or less resistant to protein and/or lipid deposits from tear film.

Medical devices such as ophthalmic lenses can generally be sub-divided into two major classes, namely hydrogels and non-hydrogels. Non-hydrogels do not absorb appreciable amounts of water, whereas hydrogels can absorb and retain water in an equilibrium state. With respect to silicone medical devices, both non-hydrogel and hydrogel silicone medical devices tend to have relatively hydrophobic, non-wettable surfaces that have a high affinity for lipids. This problem is of particular concern with contact lenses.

Those skilled in the art have recognized the need for modifying the surface of silicone ophthalmic devices, such as contact lenses and intraocular lenses, so that they are compatible with the eye. It is known that, in general, increased hydrophilicity of a contact lens surface improves the wettability of the contact lenses. This in turn is associated with improved wear comfort of contact lenses. Additionally, the surface of the lens can affect the lens's susceptibility to deposition, particularly the deposition of proteins and lipids from the tear fluid during lens wear. Accumulated deposition can cause eye discomfort or even inflammation. In the case of extended wear lenses (i.e., lenses used without daily removal of the lens before sleep), the surface is especially important, since extended wear lens must be designed for high standards of comfort and biocompatibility over an extended period of time.

Various methods of changing the surface characteristics of medical devices involve subjecting the device surfaces to a plasma or an electrical glow discharge. As one example, plasma oxidation is conducted by plasma treating the device surface in the presence of an oxidizing agent such as oxygen. As another example, a material may be deposited on or grafted on the surface of a device by plasma treating the device surface in an environment containing the material to be deposited or grafted. Combinations of plasma oxidation and plasma coating or deposition may also be performed. For example, one embodiment of U.S. Pat. No. 6,213,604 (Valint, Jr. et al.) involves: (a) subjecting the surface of a lens to a plasma oxidation reaction to create oxygen or nitrogen containing functional groups on the surface of the lens, in order to promote adhesion of the subsequent carbon coating; (b) subjecting the oxidized surface of the lens to a plasma polymerization deposition with a gas made from a diolefinic compound having 4 to 8 carbon atoms, in the absence of oxygen, thus forming a carbon layer on the surface on the lens; and (c) rendering the surface of the carbon coating hydrophilic and wettable to tear fluid by subjecting it to a second plasma oxidation.

Additionally, materials may be grafted or deposited on a device surface without the use of a plasma treatment. Generally, these methods involve contacting the surface of the device with a solution containing the material to be grafted or deposited on the lens surface. For example, the material to be deposited may include functionality that is reactive with a complementary functionality at or near the device surface. Also, the device surface may be pretreated, prior to the contacting step, so that it is has better affinity for the material to be deposited. Although in some cases the contact step may be conducted at room temperature, typically, the device and the solution are heated for an extended period to effect the grafting or deposition of the material to the device surface.

For example, another embodiment of U.S. Pat. No. 6,213,604 (Valint, Jr. et al.) involves: (a) subjecting the surface of a lens to a plasma oxidation reaction to create oxygen or nitrogen containing functional groups on the surface of the lens, in order to promote adhesion of the subsequent carbon coating; (b) subjecting the oxidized surface of the lens to a plasma polymerization deposition with a gas made from a diolefinic compound having 4 to 8 carbon atoms, in the absence of oxygen, thus forming a carbon layer on the surface on the lens; and (c) graft polymerizing a hydrophilic polymer to the lens surface. In this embodiment, step (c) involves the formation of a coating of the hydrophilic polymer on the surface without the use of plasma, and involves heating the solution containing the hydrophilic polymer for an extended period while it is in contact with the lens.

The present invention recognized that, for surface modification processes, or steps thereof, which do not employ a plasma treatment, it would be desirable to shorten the time required and/or to employ lower treatment temperatures.

### SUMMARY OF THE INVENTION

This invention provides a method of modifying the surface of a medical device, such as an ophthalmic lens. The method involves contacting a surface of the medical device with a solution containing a surface modifying agent; and subjecting the device surface and surface modifying agent to microwave radiation while the surface modifying agent is in contact with the device surface. The device may be constructed of materials such as hydrogel copolymers and silicone materials.

The surface modifying agent may be attached to the device by various mechanisms, such as covalent bonding or ionic bonding. As an example, the surface modifying agent may comprise a proton-donating wetting agent, such as an acrylic acid polymer, that complexes with the device. As another example, the surface modifying agent may comprise a reactive hydrophilic copolymer that is the polymerization product of a monomer mixture comprising a hydrophilic monomer and a monomer containing a reactive group, wherein the reactive group reacts with the device.

### DETAILED DESCRIPTION OF PREFERRED EMBODIMENTS

The method of the present invention is useful for treating a wide variety of medical devices, including both soft and rigid materials commonly used for ophthalmic lenses, such as contact lenses and intraocular lenses.

Hydrogels represent one class of materials used for many device applications, including ophthalmic lenses. Hydrogels comprise a hydrated, cross-linked polymeric systems containing water in an equilibrium state. Accordingly, hydrogels are copolymers prepared from hydrophilic monomers. In the case of silicone hydrogels, the hydrogel copolymers are generally prepared by polymerizing a mixture containing at least one device-forming silicone-containing monomer and at least one device-forming hydrophilic monomer. Either the silicone-containing monomer or the hydrophilic monomer may function as a crosslinking agent (a crosslinking agent being defined as a monomer having multiple polymerizable functionalities), or alternately, a separate crosslinking agent may be employed in the initial monomer mixture from which the hydrogel copolymer is formed. Silicone hydrogels typically have a water content between about 10 to about 80 weight percent.

Examples of useful device-forming hydrophilic monomers include: amides such as N,N-dimethylacrylamide and N,N-dimethylmethacrylamide; cyclic lactams such as N-vinyl-2-pyrrolidone; (meth)acrylated alcohols, such as 2-hydroxyethylmethacrylte and 2-hydroxyethylacrylate; and (meth)acrylated poly(alkene glycols), such as poly(diethylene glycols) of varying chain length containing monomethacrylate or dimethacrylate end caps. Still further examples are the hydrophilic vinyl carbonate or vinyl carbamate monomers disclosed in U.S. Pat. Nos. 5,070,215, and the hydrophilic oxazolone monomers disclosed in U.S. Pat. No. 4,910,277, the disclosures of which are incorporated herein by reference. Other suitable hydrophilic monomers will be apparent to one skilled in the art.

As mentioned, one preferred class of medical device materials are silicone hydrogels. In this case, the initial device-forming monomer mixture further comprises a silicone-containing monomer.

Applicable silicone-containing monomeric materials for use in the formation of silicone hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Examples of applicable silicon-containing monomers include bulky polysiloxanylalkyl(meth)acrylic monomers. An example of bulky polysiloxanylalkyl(meth)acrylic monomers are represented by the following Formula I:

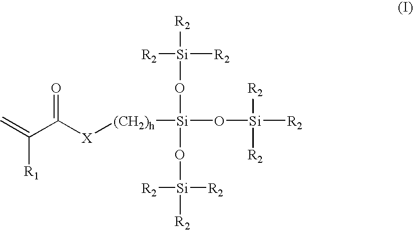

wherein:

X denotes —O— or —NR—;

each R₁ independently denotes hydrogen or methyl;

each R₂ independently denotes a lower alkyl radical, phenyl radical or a group represented by

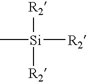

wherein each R′₂ independently denotes a lower alkyl or phenyl radical; and h is 1 to 10. One preferred bulky monomer is methacryloxypropyl tris(trimethyl-siloxy)silane or tris(trimethylsiloxy)silylpropyl methacrylate, sometimes referred to as TRIS.

Another class of representative silicon-containing monomers includes silicone-containing vinyl carbonate or vinyl carbamate monomers such as: 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(tri-methylsiloxy)silyl]propyl vinyl carbamate; 3-[tris(trimethylsiloxy)silyl]propyl allyl carbamate; 3-[tris(trimethylsiloxy)silyl]propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II:

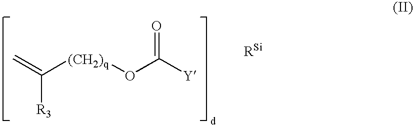

wherein:

Y′ denotes —O—, —S— or —NH—;

R^{Si} denotes a silicone-containing organic radical;

R₃ denotes hydrogen or methyl;

d is 1, 2, 3 or 4; and q is 0 or 1.

Suitable silicone-containing organic radicals R^{Si} include the following:

—(CH₂)ₙ′Si[(CH₂)ₘ′CH₃]₃ ;

—(CH₂)ₙ′Si[OSi(CH₂)ₘ′CH₃]₃ ;

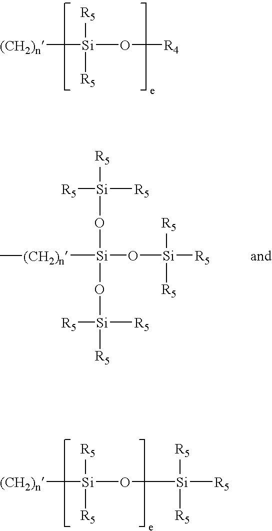

wherein:

R₄ denotes

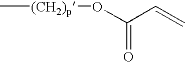

wherein p′ is 1 to 6;

R₅ denotes an alkyl radical or a fluoroalkyl radical having 1 to 6 carbon atoms;

e is 1 to 200; n′ is 1, 2, 3 or 4; and m′ is 0, 1, 2, 3, 4 or 5.

An example of a particular species within Formula II is represented by Formula III.

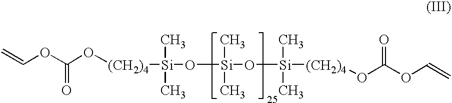

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicone urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicone urethane monomers are represented by Formulae IV and V:

E(*D*A*D*G)ₐ*D*A*D*E′; or (IV)

E(*D*G*D*A)ₐ*D*G*D*E′; (V)

wherein:

D denotes an alkyl diradical, an alkyl cycloalkyl diradical, a cycloalkyl diradical, an aryl diradical or an alkylaryl diradical having 6 to 30 carbon atoms;

G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;

denotes a urethane or ureido linkage;

a is at least 1;

A denotes a divalent polymeric radical of Formula VI:

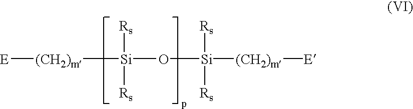

wherein:

each R_{S} independently denotes an alkyl or fluoro-substituted alkyl group having 1 to 10 carbon atoms which may contain ether linkages between carbon atoms;

m′ is at least 1; and

p is a number which provides a moiety weight of 400 to 10,000;

each of E and E′ independently denotes a polymerizable unsaturated organic radical represented by Formula VII:

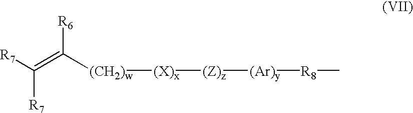

wherein:

R₆ is hydrogen or methyl;

R₇ is hydrogen, an alkyl radical having 1 to 6 carbon atoms, or a —CO—Y—R₉ radical wherein Y is —O—, —S— or —NH—;

R₈ is a divalent alkylene radical having 1 to 10 carbon atoms;

R₉ is a alkyl radical having 1 to 12 carbon atoms;

X denotes —CO— or —OCO—;

Z denotes —O— or —NH—;

Ar denotes an aromatic radical having 6 to 30 carbon atoms;

w is 0 to 6; x is 0 or 1; y is 0 or 1; and z is 0 or 1.

A more specific example of a silicone-containing urethane monomer is represented by Formula (VIII):

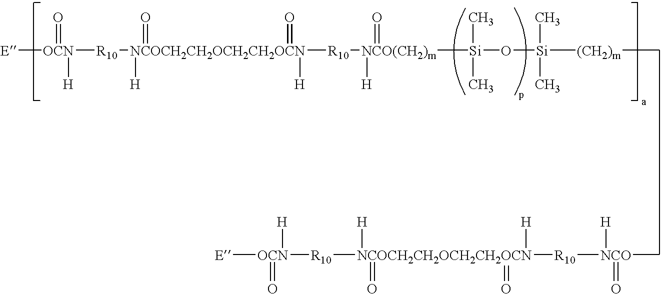

wherein m is at least I and is preferably 3 or 4, a is at least 1 and preferably is 1, p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

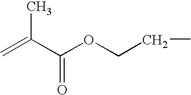

A preferred silicone hydrogel material comprises (based on the initial monomer mixture that is copolymerized to form the hydrogel copolymeric material) 5 to 50 percent, preferably 10 to 25, by weight of one or more silicone macromonomers, 5 to 75 percent, preferably 30 to 60 percent, by weight of one or more polysiloxanylalkyl (meth)acrylic monomers, and 10 to 50 percent, preferably 20 to 40 percent, by weight of a hydrophilic monomer. In general, the silicone macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

Specific examples of substrate materials useful in the present invention are taught in U.S. Pat. No. 5,908,906 to Künzler et al.; U.S. Pat. No. 5,714,557 to Künzler et al.; U.S. Pat. No. 5,710,302 to Künzler et al.; U.S. Pat. No. 5,708,094 to Lai et al.; U.S. Pat. No. 5,616,757 to Bambury et al.; U.S. Pat. No. 5,610,252 to Bambury et al.; U.S. Pat. No. 5,512,205 to Lai; U.S. Pat. No. 5,449,729 to Lai; U.S. Pat. No. 5,387,662 to Künzler et al. and U.S. Pat. No 5,310,779 to Lai; the disclosures of which are incorporated herein by reference.

With respect to intraocular lenses (IOLs), poly(methyl methacrylate) (PMMA), which is a rigid, glassy polymer, was the most widely used material for decades. In recent years, softer, more flexible IOL implants have gained in popularity years due to their ability to be compressed, folded, rolled or otherwise deformed. Such softer IOL implants may be deformed prior to insertion thereof through an incision in the cornea of an eye. Following insertion of the IOL in an eye, the IOL returns to its original pre-deformed shape due to the memory characteristics of the soft material. Softer, more flexible IOL implants as just described may be implanted into an eye through an incision that is much smaller, i.e., less than 4.0 mm, than that necessary for more rigid IOLs, i.e., 5.5 to 7.0 mm. A larger incision is necessary for more rigid IOL implants because the lens must be inserted through an incision in the cornea slightly larger than the diameter of the inflexible IOL optic portion. Accordingly, more rigid IOL implants have become less popular in the market since larger incisions have been found to be associated with an increased incidence of postoperative complications, such as induced astigmatism.

With recent advances in small-incision cataract surgery, increased emphasis has been placed on developing soft, foldable materials suitable for use in the manufacture of IOL implants. In general, the materials of current commercial IOLs fall into one of three categories: silicone, hydrophilic acrylic and hydrophobic acrylic. These categories include hydrogel and non-hydrogel materials, and many examples of IOL materials are well-known to those skilled in the art.

This invention is applicable to a wide variety of surface modification processes. Generally, the method of this invention involves contacting a device surface with a solution containing the surface modifying agent. The device is exposed to microwave radiation while the surface modifying agent is in contact with the device. The term “microwave radiation” denotes radiation predominantly in the frequency range of 1 to 40 GHz. The microwave radiation facilitates attachment of the surface modifying agent to the device surface. The surface modifying agent may be attached to the lens surface by various means, including: formation of a covalent bond between a reactive group on the surface modifying agent and a complementary reactive group on or near the surface of the device; ionic bonding between such reactive groups; hydrogen bonding between such reactive groups; complexation between a surface modifying agent having a proton donating moiety and a device having relatively proton donating moieties; as well as other methods of attachment.

Generally, various prior surface modification methods involve heating the solution containing the surface modifying agent, such as by autoclaving, but the present invention can significantly reduce cycle time. Preferably, microwave radiation is applied for less than 10 minutes, more preferably less than 5 minutes, and most preferably less than two minutes.

Various microwave apparatus are commercially available for industrial and scientific applications. A representative apparatus is available under the tradename Magnitron FS-14 EVP (Litton, Memphis, Tenn., USA ), although persons skilled in the art will readily know of other commercially available apparatus.

As an example, a coating layer may be formed according to the method described in U.S. Pat. No. 6,428,839 (Kunzler et al.), the disclosures of which is incorporated herein by reference. Generally, this method employs poly(acrylic) acid (PAA) surface complexation. Hydrogel contact lens copolymers containing polymerized hydrophilic monomers having relatively strong proton donating moieties, for example DMA or NVP, are treated with water-based solutions containing PAA or PAA co-polymers, acting as wetting agents, to render a lubricious, stable, highly wettable PAA-based surface coating. Alternately, other proton-donating wetting agents besides PAA-containing agents may be employed, although generally, coating materials containing carboxylic acid functionality are preferred. In this method, no additional oxidative surface treatment such as corona discharge or plasma oxidation is required. Surface coating materials include poly(vinylpyrrolidinone(VP)-co-acrylic acid(AA)), poly(methylvinylether-alt-maleic acid), poly(acrylic acid-graft-ethyleneoxide), poly(AA-co-methacrylic acid), poly(acrylamide-co-AA), poly(AA-co-maleic), and poly(butadiene-maleic acid). Particularly preferred polymers are characterized by acid contents of at least about 30 mole percent, preferably at least about 40 mole percent.

Solvents useful in the surface treatment (contacting) step of this method include solvents that readily solubilize proton donating solubes such as carboxylic acids. Preferred solvents include tetrahydrofuran (THF), acetonitrile, N,N-dimethyl formamide (DMF), and water. The surface treatment solution is preferably acidified before the contact step. The pH of the solution is suitably less than 7, preferably less than 5 and more preferably less than 4. For a discussion of the theory underlying the role of pH in complexation reactions in general, see Advances in Polymer Science, published by Springer-Verlag, Editor H. J. Cantow, et al, V45, 1982, pages 17-63.

The surface treatment generally consists of immersing the lens in the PAA-containing solution. However, instead of subjecting the solution and lens to several autoclave cycles, such as described in U.S. Pat. No. 6,428,839, the solution and lens are exposed to microwave radiation for a much shorter period of time. The lenses are then rinsed in distilled water.

The resultant contact lens has its external surface coated with the PAA coating layer, such coating being hydrophilic, wettable and lubricious.

As another example, this invention is applicable to the coating method described in U.S. application Ser. No. 10/187,056 (filed Jun. 28, 2002), the disclosure of which is incorporated herein by reference. Generally, this method involves surface modification of medical devices, particularly, IOLs, with one or more reactive, hydrophilic polymers. The reactive, hydrophilic polymers are copolymers of at least one hydrophilic monomer and at least one monomer that contains reactive chemical functionality. The hydrophilic monomers can be aprotic types such as N,N-dimethylacrylamide and N-vinylpyrrolidone or protic types such as methacrylic acid and 2-hydroxyethyl methacrylate. The monomer containing reactive chemical functionality can be an epoxide-containing monomer, such as glycidyl methacrylate. The hydrophilic monomer and the monomer containing reactive chemical functionality are copolymerized at a desired molar ratio thereof. The hydrophilic monomer serves to render the resultant copolymer hydrophilic. The monomer containing reactive chemical functionality provides a reactive group that can react with the lens surface. In other words, this resultant copolymer contains the reactive chemical functionality that can react with complementary functional groups at or near the lens surface.

According to this embodiment of the present invention, the device is contacted with a solution containing the reactive, hydrophilic copolymer, for example, by immersing the device in this solution. Then, instead of autoclaving, the solution and device are subjected to microwave radiation, thereby reducing considerably the treatment time.

As another example, a coating layer may be formed on the device surface according to the method described in U.S. Pat. No. 6,200,626, the disclosure of which is incorporated herein by reference. Generally, this method involves: (a) subjecting an oxidized surface of the lens to a plasma-polymerization deposition with an C1 to C10 saturated or unsaturated hydrocarbon to form a polymeric carbonaceous primary coating (or “carbon layer”) on the lens surface; and (b) grafting a hydrophilic monomer onto the carbon layer by free-radical polymerization of the monomers to form a hydrophilic, biocompatible, secondary polymeric coating. Specifically, according to this invention, the grafting of step (b) is facilitated by employing microwave radiation.

Step (a) involves a standard plasma oxidation and deposition processes (also referred to as “electrical glow discharge processes”) to provide a thin, durable surface on the lens prior to the covalently bonded grafting of the hydrophilic polymeric coating in step (b). Such plasma processes are known in the art, and examples are provided in U.S. Pat. Nos. 4,143,949; 4,312,575; and 5,464,667, the disclosures of which are incorporated herein by reference. Plasma surface treatments involve passing an electrical discharge through a gas at low pressure. The electrical discharge may be at radio frequency (typically 13.56 MHz), although microwave and other frequencies can be used. Electrical discharges produce ultraviolet (UV) radiation, in addition to being absorbed by atoms and molecules in their gas state, resulting in energetic electrons and ions, atoms (ground and excited states), molecules and radicals. Thus, a plasma is a complex mixture of atoms and molecules in both ground and excited states, which reach a steady state after the discharge is begun. The circulating electrical field causes these excited atoms and molecules to collide with one another as well as the walls of the chamber and the surface of the material being treated.

The deposition of a coating from a plasma onto the surface of a material has been shown to be possible from high-energy plasmas without the assistance of sputtering (sputter-assisted deposition). Monomers can be deposited from the gas phase and polymerized in a low-pressure atmosphere (0.005 to 5 torr, preferably 0.01 to 1.0 torr) onto a substrate utilizing continuous or pulsed plasmas, suitably as high as about 1000 watts. A modulated plasma, for example, may be applied 100 milliseconds on then off. In addition, liquid nitrogen cooling has been utilized to condense vapors out of the gas phase onto a substrate and subsequently use the plasma to chemically react these materials with the substrate. However, plasmas generally do not require the use of external cooling or heating to cause the desired deposition.

Preferably, step (a) is preceded by subjecting the surface of the lens surface to a plasma oxidation reaction so as to more effectively bond the polymerized hydrocarbon coating to the lens and to resist delamination and/or cracking of the surface coating from the lens upon lens hydration. Thus, for example, if the lens is ultimately made from a hydrogel material that is hydrated (wherein the lens typically expands by ten to about twenty percent), the coating remains intact and bound to the lens, providing a more durable coating which is resistant to delamination and/or cracking. Such an oxidation of the lens may be accomplished in an atmosphere composed of an oxidizing media. It is preferred that a relatively “strong” oxidizing plasma is utilized for this oxidation, for example ambient air drawn through a five percent (5%) hydrogen peroxide solution. As an example, plasma oxidation may be carried out at an electric discharge frequency of 13.56 Mhz, preferably between about 20 to 500 watts at a pressure of about 0.1 to 1.0 torr, preferably for about 10 seconds to about 10 minutes or more, more preferably about 1 to 10 minutes. The contact lens can alternatively be pretreated by providing an aminated surface, by subjecting the lens to an ammonia or an aminoalkane plasma. Those skilled in the art will recognize other methods of improving or promoting adhesion for bonding of the subsequent carbon layer. For example, plasma with an inert gas may also improve bonding.

Then, in step (a), a thin hydrocarbon coating is deposited on the lens, and in step (b), the carbon surface is exposed to, and reacted with, the hydrophilic monomer, or mixture of monomers including the hydrophilic monomer, under free-radical polymerization conditions, resulting in a hydrophilic polymer coating attached to the carbon surface.

In step (a), the lens surface is subjected to the plasma polymerization reaction in a hydrocarbon atmosphere to form a polymeric surface on the lens. Any hydrocarbon capable of polymerizing in a plasma environment may be utilized; however, the hydrocarbon generally should be in a gaseous state during polymerization and have a boiling point below about 200° C. at one atmosphere. Preferred hydrocarbons include aliphatic compounds having from 1 to about 15 carbon atoms, including both saturated and unsaturated aliphatic compounds. Examples include, but are not limited to, C1 to C15, preferably C1 to C10 alkanes, alkenes, or alkynes such as methane, ethane, propane, butane, pentane, hexane, ethylene, propylene, butylene, cyclohexane, pentene, acetylene. Also, C1 to C8 aromatics such as benzene, styrene, methylstyrene, and the like may be employed. As is known in the art, such hydrocarbon groups may be unsubstituted or substituted so long as they are capable of forming a plasma. Various combinations of different hydrocarbons may also be used.

The use of C1 to C4 hydrocarbons for the purpose of carbon-coating substrates is advantageous for its controllability in terms of thickness, deposition rate, hardness, etc. However, with respect to hydrogel materials, the C4 to C8 hydrocarbons (for example, butane, butene, isobutylene, and 1,3-butadiene) are advantageous, due to being relatively more flexible than coatings made from C1 to C3 hydrocarbons such as methane. Diolefins such as 1,3-butadiene or isoprene are particularly advantageous, resulting in coatings that are both flexible and expandable in water. More flexible coatings are especially preferred for “high-water” contact lenses that expand considerably upon hydration.

The hydrocarbon coating can be deposited from plasma, for example, in a low-pressure atmosphere (about 0.001 to 5 torr) at a radio frequency of 13.56 Mhz, at about 10 to 1000 watts, preferably 20-400 watts in about 30 seconds to 10 minutes or more, more preferably 30 seconds to 3 minutes. Other plasma conditions may be suitable as will be understood by the skilled artisan, for example, using pulsed plasma.

If the hydrocarbon coating provided is too thick, it can cause a haziness, resulting in a cloudy lens. Furthermore, excessively thick coatings can interfere with lens hydration due to differences in expansion between the lens and the coating, causing the lens to rip apart. Therefore, the thickness of the hydrocarbon layer should be less than about 500 Angstroms, preferably between about 25 and 500 Angstroms, more preferably 50 to 200 Angstroms, as determined by XPS analysis.

To form the polymer coating in step (b), an initiator may be employed to cause the ethylenically-unsaturated monomer to react with the surface. In any case, the carbon layer must be rendered reactive (activated) to promote covalent attachment. One advantage of diolefins to form the carbon layer is that unsaturated sites for the initiation of graft polymerization are already present. When employing other hydrocarbons to form the carbon layer, an activator or initiator may be employed to speed the free-radical graft polymerization of the surface. Alternately, conventional techniques for the initiation of graft polymerization may be applied to the carbon layer to create peroxy or other functional groups that can also initiate graft polymerization. For example, it is known in the art that various vinyl monomers can be graft polymerized onto polymer substrates which have been first treated with ionizing radiation in the presence of oxygen or with ozone to form peroxy groups on the surface of said substrate. See U.S. Pat. Nos. 3,008,920 and 3,070,573, for instance, for ozonization of the substrate. Alternatively, a carbon layer formed by plasma may already contain radicals that when exposed to air, form peroxide groups that decompose to oxygen radicals. Additional plasma/corona treatment is also capable of forming radicals for reaction with ethylenically-unsaturated monomers or polymers. Still another way to promote graft polymerization is to plasma treat the substrate, for example with argon or helium in plasma form, to form free radicals on its outmost surfaces, then contacting these radicals with oxygen to form hydroperoxy groups from the free radicals, followed by graft polymerizing ethylenically unsaturated monomers onto the surface.

The grafting polymer may be formed by using an aqueous solution of the ethylenically unsaturated monomer or mixture of monomers capable of undergoing graft addition polymerization onto the surface of the substrate. In those cases where one or more of the monomers is not appreciably soluble in water, a cosolvent such as tert-butyl alcohol may be used to enhance the solubility of the monomer in the aqueous graft polymerization system. The graft polymer may be the reaction product of a mixture of monomers comprising one or more hydrophilic monomers, including the aforementioned hydrophilic monomers employed as hydrogel copolymer lens-forming monomers. Specific examples of hydrophilic monomers for grafting to the carbon layer include aprotic types: acrylamides, such as N,N-dimethylacrylamide (DMA); vinyl lactams, such as N-vinylpyrrolidinone (NVP); and (meth)acrylated poly(alkylene oxides) such as methoxypolyoxyethylene methacrylates. Other specific examples include protic types: (meth)acrylic acid; and hydroxyalkyl (meth)acrylates, such as hydroxyethyl methacrylate (Hema). Hydrophilic monomers may also include zwitterions such as N,N-dimethyl-N-methacryloxyethyl-N-(3-sulfopropyl)-ammonium betain (SPE) and N,N-dimethyl-N-methacrylamidopropyl-N-(3-sulfopropyl)-ammonium betain (SPP). Optionally, some hydrophobic monomers may also be included with the hydrophilic monomer to impart desired properties such as resistance to lipid or protein deposition. Examples of hydrophobic monomers are alkyl methacrylate, fluorinated alkyl methacrylates, long-chain acrylamides such as octyl acrylamide, and the like. This monomeric mixture may be applied to the contact lens by dipping the front surface of the lens in the monomer mixture, or by spraying this mixture on the lens surface.

The graft polymerization of step (b) is typically carried out in the presence of a solvent. Determination of reactivity ratios for copolymerization are disclosed in Odian, Principles of Polymerization, 2nd Ed., John Wiley & Sons, p. 425-430 (1981), the disclosure of which is incorporated by reference herein. For example, the contact lens is contacted with the mixture of the reactive monomers in a suitable medium, for example, an aprotic solvent such as acetonitrile. Then, the lens and solution are exposed to microwave radiation to facilitate the graft polymerization. Suitable solvents are those which dissolve the monomers, including: water; alcohols such as lower alkanols, for example, ethanol and methanol; carboxamides such as dimethylformamide; dipolar aprotic solvents such as dimethyl sulfoxide or methyl ethyl ketone; ketones such as acetone or cyclohexanone; hydrocarbons such as toluene; ethers such as THF, dimethoxyethane or dioxane; halogenated hydrocarbons such as trichloroethane, and also mixtures of suitable solvents, for example mixtures of water and an alcohol, for example a water/ethanol or water/methanol mixture.

To further promote the free-radical grafting, the lens substrate may optionally be immersed in a first solution containing an initiator followed by a immersion of the substrate in a second solution containing the hydrophilic monomer or mixture thereof. Typical polymerization initiators include free-radical-generating polymerization initiators of the type illustrated by acetyl peroxide, lauroyl peroxide, decanoyl peroxide, coprylyl peroxide, benzoyl peroxide, tertiary butyl peroxypivalate, sodium percarbonate, tertiary butyl peroctoate, and azobis-isobutyronitrile (AIBN). The curing process will of course depend upon the initiator used and the physical characteristics of the comonomer mixture such as viscosity. If an initiator is employed, it is typically present at a level within the range of 0.01 to 2 weight percent of the monomer mixture.

As another example, the coating layer may be formed according to the method described in U.S. published application Ser. No. 2002-0102415-A1 or PCT publication WO 00/71613), the disclosures of which are incorporated herein by reference. Generally, this method involves: (a) subjecting an oxidized surface of the lens to a plasma-polymerization deposition with an C1 to C10 saturated or unsaturated hydrocarbon to form a polymeric carbonaceous primary coating (or “carbon layer”) on the lens surface; (b) forming reactive functionalities on the surface of the carbon layer; and (c) attaching hydrophilic polymer chains to the carbon layer by reacting the reactive functionalities on the carbon layer with complementary isocyanate or ring-opening reactive functionalities along a reactive hydrophilic polymer. More specifically, the attachment of the hydrophilic polymer chains to the carbon layer is effected by microwave radiation.

Step (a) of this coating process is similar to step (a) in the immediately aforementioned coating process, and similarly, is preferably preceded by subjecting the surface of the lens to a plasma-oxidation reaction so as to more effectively bond the polymerized hydrocarbon coating to the lens. In step (b), reactive functionalities are formed on the surface of the carbon layer to form the point of attachment for hydrophilic polymer chains. In step (c), the functionalized carbon surface is exposed to, and reacted with, hydrophilic reactive polymers, resulting in hydrophilic polymer chains attached to the carbon surface, rendering the carbon coating of step (a) hydrophilic. Any complementary reactive functionalities on the hydrophilic reactive polymer that remain unreacted, after attachment to the carbon surface at one or more locations, may be hydrolyzed as explained below. Preferably, on average the hydrophilic polymers become attached to the substrate surface at a plurality of points, therefore forming one or more loops on the surface.

Various methods are known in the art to attach a polymer chain to a carbon layer, including plasma oxidation or other means to provide surface reactive functional groups that can react with the polymer. Preferably, a nitrogen-containing gas is used to aminate, or form amine groups on, the carbon layer. However, oxygen or sulfur containing gases may alternately be used to form oxygen or sulfur containing groups, for example hydroxy or sulfide groups, on the carbon layer. Thus, the carbon layer is rendered reactive (functionalized) to promote the covalent attachment of the hydrophilic polymer to the surface.

To create an aminated carbon layer, the oxidation preferably utilizes a gas composition comprising an oxidizing media such as ammonia, ethylene diamine, C1 to C8 alkyl amine, hydrazine, or other oxidizing compounds. Preferably, the oxidation of the hydrocarbon layer is performed for a period of about 10 seconds to 10 minutes or more, more preferably 1 to 10 minutes, a discharge frequency of 13.56 Mhz at about 10 to 1000 watts, preferably 20 to 500 watts and about 0.1 to 1.0 torr.

The hydrophilic polymer, which is attached to the reactive functionalities on the carbon coating, may be the reaction product of monomers comprising one or more non-reactive hydrophilic monomers and one or more reactive functional monomers. In this case, the reactive functional monomeric unit will react with complementary reactive functionalities on the surface provided by the previous plasma oxidation. Such reactive functional monomers may include monomers containing one or more of the following groups: cyanate (—CNO); or various ring-opening reactive groups, for example, azlactone, epoxy, acid anhydrides, and the like.

The hydrophilic reactive polymers may be homopolymers or copolymers comprising reactive monomeric units that contain either an isocyanate or a ring-opening reactive functionality optionally. Although these reactive monomeric units may also be hydrophilic, the hydrophilic reactive polymer may also be a copolymer of reactive monomeric units copolymerized with one or more of various non-reactive hydrophilic monomeric units. Lesser amounts of hydrophobic monomeric units may optionally be present in the hydrophilic polymer, and in fact may be advantageous in providing a thicker coating by promoting the aggregation of the hydrophilic reactive polymer in solution. The ring-opening monomers include azlactone-functional, epoxy-functional and acid-anhydride-functional monomers.

Mixtures of hydrophilic reactive polymers may be employed. For example, the hydrophilic polymer chains attached to the carbonaceous layer may be the result of the reaction of a mixture of polymers comprising (a) a first hydrophilic reactive polymer having reactive functionalities in monomeric units along the hydrophilic polymers complementary to reactive functionalities on the carbonaceous layer and, in addition, (b) a second hydrophilic reactive polymer having supplemental reactive functionalities that are reactive with the first hydrophilic reactive polymer. A mixture comprising an epoxy-functional polymer with an acid-functional polymer, either simultaneously or sequentially applied to the substrate to be coated, have been found to provide relatively thick coatings.

Preferably the hydrophilic reactive polymers comprise 1 to 100 mole percent of reactive monomeric units, more preferably 5 to 50 mole percent, most preferably 10 to 40 mole percent. The polymers may comprise 0 to 99 mole percent of non-reactive hydrophilic monomeric units, preferably 50 to 95 mole percent, more preferably 60 to 90 mole percent (the reactive monomers, once reacted may also be hydrophilic, but are by definition mutually exclusive with the monomers referred to as hydrophilic monomers which are non-reactive). Other monomeric units which are hydrophobic optionally may also be used in amounts up to about 35 mole percent, preferably 0 to 20 mole percent, most preferably 0 to 10 mole percent. Examples of hydrophobic monomers are alkyl methacrylate, fluorinated alkyl methacrylates, long-chain acrylamides such as octyl acrylamide, and the like. Hydrophilic monomers may be aprotic types, such as acrylamides vinyl lactones, and poly(alkylene oxides), or may be protic types such as (meth)acrylic acid or hydroxyalkyl (meth)acrylates. Hydrophilic monomers may also include zwitterions.

The weight average molecular weight of the hydrophilic reactive polymer may suitably range from about 200 to 1,000,000, preferably from about 1,000 to 500,000, most preferably from about 5,000 to 100,000.

As mentioned above, the hydrophilic reactive polymer may comprise monomeric units derived from azlactone-functional, epoxy-functional and acid-anhydride-functional monomers. For example, an epoxy-functional hydrophilic reactive polymer for coating a lens can be a copolymer containing glycidyl methacrylate (GMA) monomeric units which will react with amine reactive functionalities or the like on the carbon layer. Preferred examples of anhydride-functional hydrophilic reactive polymers comprise monomeric units derived from monomers such as maleic anhydride and itaconic anhydride.

In general, epoxy-functional reactive groups or anhydride-functional reactive groups in the hydrophilic reactive polymer react with the primary amine (—NH₂) groups or other reactive functionalities formed by plasma-oxidation on the carbon layer. Although amine reactive functionalities are preferred, oxygen-containing groups may be employed, preferably in the presence of an acidic catalyst such as 4-dimethylaminopyridine, to speed the reaction at room temperature, as will be understood by the skilled chemist. In general, azlactone or isocyanate-functional groups in the hydrophilic reactive polymers may similarly react with amines or hydroxy radicals, or the like, on the carbon layer.

Preferably, preformed (non-polymerizable) hydrophilic polymers containing repeat units derived from at least one ring-opening monomer or isocyanate-containing monomer are covalently reacted with reactive groups on the surface of the medical device such as a contact lens substrate. Typically, the hydrophilic reactive polymers are attached to the substrate at one or more places along the chain of the polymer. After attachment, any unreacted reactive functionalities in the hydrophilic reactive polymer may be hydrolyzed to a non-reactive moiety.

The hydrophilic reactive polymers are synthesized in a known manner from the corresponding monomers (the term monomer again also including a macromonomer) by a polymerization reaction customary to the person skilled in the art. Typically, the hydrophilic reactive polymers or chains are formed by: (1) mixing the monomers together; (2) adding a polymerization initiator; (3) subjecting the monomer/initiator mixture to a source of ultraviolet or actinic radiation and curing said mixture. Typical polymerization initiators include free-radical-generating polymerization initiators of the type illustrated by acetyl peroxide, lauroyl peroxide, decanoyl peroxide, coprylyl peroxide, benzoyl peroxide, tertiary butyl peroxypivalate, sodium percarbonate, tertiary butyl peroctoate, and azobis-isobutyronitrile (AIBN). Ultraviolet free-radical initiators illustrated by diethoxyacetophenone can also be used. The curing process will of course depend upon the initiator used and the physical characteristics of the comonomer mixture such as viscosity. In any event, the level of initiator employed will vary within the range of 0.01 to 2 weight percent of the mixture of monomers.

The polymerization to form the hydrophilic reactive polymer can be carried out in the presence of a solvent. Suitable solvents include water, alcohols such as lower alkanols, for example, ethanol and methanol; carboxamides such as dimethylformamide; dipolar aprotic solvents such as dimethyl sulfoxide or methyl ethyl ketone; ketones such as acetone or cyclohexanone; hydrocarbons such as toluene; ethers such as THF, dimethoxyethane or dioxane; halogenated hydrocarbons such astrichloroethane, and also mixtures of suitable solvents, for example mixtures of water and an alcohol, for example a water/ethanol or water/methanol mixture.

The carbon-coated contact lens may be exposed to the hydrophilic reactive polymer by immersing the lens substrate in a solution containing the polymer or by spraying the solution on the lens surface.

As indicated above, this coating method involves attaching reactive hydrophilic polymers to a functionalized carbon coating, which polymers comprise isocyanate-containing monomeric units or ring-opening monomeric units. The ring-opening reactive monomer may be an azlactone group represented by the following formula:

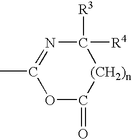

wherein R³ and R⁴ independently can be an alkyl group having 1 to 14 carbon atoms, a cycloalkyl group having 3 to 14 carbon atoms, an aryl group having 5 to 12 ring atoms, an arenyl group having 6 to 26 carbon atoms, and 0 to 3 heteroatoms non-peroxidic selected from S, N, and O, or R³ and R⁴ taken together with the carbon to which they are joined can form a carbocyclic ring containing 4 to 12 ring atoms, and n is an integer 0 or 1. Such monomeric units are disclosed in U.S. Pat. No. 5,177,165 to Valint et al.

The ring structure of such reactive functionalities is susceptible to nucleophilic ring-opening reactions with complementary reactive functional groups on the surface of the carbon layer or substrate being treated. For example, the azlactone functionality can react with primary amines, hydroxyl radicals or the like formed by plasma oxidation of the carbon layer, as mentioned above, to form a covalent bond between the substrate and the hydrophilic reactive polymer at one or more locations along the polymer. A plurality of attachments can form a series of polymer loops on the substrate, wherein each loop comprises a hydrophilic chain attached at both ends to the substrate.

Azlactone-functional monomers for making the hydrophilic reactive polymer can be any monomer, prepolymer, or oligomer comprising an azlactone functionality of the above formula in combination with a vinylic group on an unsaturated hydrocarbon to which the azlactone is attached. Preferably, azlactone-functionality is provided in the hydrophilic polymer by 2-alkenyl azlactone monomers. The 2-alkenyl azlactone monomers are known compounds, their synthesis being described, for example, in U.S. Pat. Nos. 4,304,705; 5,081,197; and 5,091,489 (all Heilmann et al.) the disclosures of which are incorporated herein by reference. Suitable 2-alkenyl azlactones include:

2-ethenyl-1,3-oxazolin-5-one,

2-ethenyl-4-methyl-1,3-oxazolin-5-one,

2-isopropenyl-1,3-oxazolin-5-one,

2-isopropenyl-4-methyl-1,3-oxazolin-5-one,

2-ethenyl-4,4-dimethyl-1,3-oxazolin-5-one,

2-isopropenyl-4-dimethyl-1,3-oxazolin-5-one,

2-ethenyl-4-methyl-ethyl-1,3-oxazolin-5-one,

2-isopropenyl-4-methyl-4-butyl-1,3-oxazolin-5-one,

2-ethenyl-4,4-dibutyl-1,3-oxazolin-5-one,

2-isopropenyl-4-methyl-4-dodecyl-1,3-oxazolin-5-one,

2-isopropenyl-4,4-diphenyl-1,3-oxazolin-5-one,

2-isopropenyl-4,4-pentamethylene-1,3-oxazolin-5-one,

2-isopropenyl-4,4-tetramethylene-1,3-oxazolin-5-one,

2-ethenyl-4,4-diethyl-1,3-oxazolin-5-one,

2-ethenyl-4-methyl-4-nonyl-1,3-oxazolin-5-one,

2-isopropenyl-methyl-4-phenyl-1,3-oxazolin-5-one,

2-isopropenyl-4-methyl-4-benzyl-1,3-oxazolin-5-one, and

2-ethenyl-4,4-pentamethylene-1,3-oxazolin-5-one.

More preferably, the azlactone monomers are a compound represented by the following general formula: where R¹ and R² independently denote a hydrogen atom or a lower alkyl radical with one to

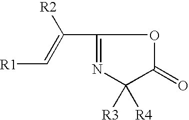

six carbon atoms, and R³ and R⁴ independently denote alkyl radicals with one to six carbon atoms or a cycloalkyl radical with five or six carbon atoms. Specific examples include 2-isopropenyl-4,4-dimethyl-2-oxazolin-5-one (IPDMO), 2-vinyl-4,4-dimethyl-2-oxazolin-5-one (VDMO), spiro-4′-(2′-isopropenyl-2′-oxazolin-5-one) cyclohexane (IPCO), cyclohexane-spiro-4′-(2′-vinyl-2′-oxazol-5′-one) (VCO), and 2-(-1-propenyl)-4,4-dimethyl-oxazol-5-one (PDMO) and the like.

As indicated above, these ring-opening compounds can be copolymerized with hydrophilic and/or hydrophobic comonomers to form hydrophilic reactive polymers. After attachment to the desired substrate, any unreacted oxazolinone groups may then be hydrolyzed in order to convert the oxazolinone components into amino acids. In general, the hydrolysis step will follow the general reaction of:

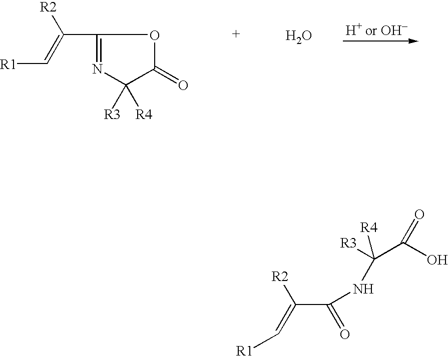

The carbon-carbon double bond between the R¹ and R² radicals is shown unreacted, but the reaction can take place when copolymerized into a polymer.

Non-limiting examples of comonomers useful to be copolymerized with azlactone functional moieties to form the hydrophilic reactive polymers used to coat a medical device include those mentioned above, preferably dimethylacrylamide, hydroxyethyl methacrylate (HEMA), and/or N-vinylpyrrolidone.

Such azlactone-functional monomers can be copolymerized with other monomers in various combinations of weight percentages. Using a monomer of similar reactivity ratio to that of an azlactone monomer will result in a random copolymer. Determination of reactivity ratios for copolymerization are disclosed in Odian, Principles of Polymerization, 2nd Ed., John Wiley & Sons, p. 425-430 (1981), the disclosure of which is incorporated by reference herein. Alternatively, use of a comonomer having a higher reactivity to that of an azlactone will tend to result in a block copolymer chain with a higher concentration of azlactone-functionality near the terminus of the chain.

Although not as preferred as monomers, azlactone-functional prepolymers or oligomers having at least one free-radically polymerizable site can also be utilized for providing azlactone-functionality in the hydrophilic reactive polymer according to the present invention. Azlactone-functional oligomers, for example, are prepared by free radical polymerization of azlactone monomers, optionally with comonomers as described in U.S. Pat. Nos. 4,378,411 and 4,695,608, incorporated by reference herein. Non-limiting examples of azlactone-functional oligomers and prepolymers are disclosed in U.S. Pat. Nos. 4,485,236 and 5,081,197 and European Patent Publication 0 392 735, all incorporated by reference herein.

Alternately, the ring-opening reactive group in the hydrophilic reactive polymer may be an epoxy functionality. The preferred epoxy-functional monomer is an oxirane-containing monomer such as glycidyl methacrylate, 4-vinyl-1-cyclohexene-1,2-epoxide, or the like, although other epoxy-containing monomers may be used. Exemplary comonomers are N,N-dimethylacrylamide and fluorinated monomers such as octafluoropentylmethacrylate.

As another example, the coating layer may be formed according to the method described in U.S. Pat. No. 6,213,604, the disclosure of which is incorporated herein by reference. Generally, this method involves: (a) subjecting the surface of the lens to a plasma oxidation reaction to create oxygen or nitrogen containing functional groups on the surface of the lens, in order to promote adhesion of the subsequent carbon coating; (b) subjecting the oxidized surface of the lens to a plasma polymerization deposition with a gas made from a diolefinic compound having 4 to 8 carbon atoms, in the absence of oxygen, thus forming a carbon layer on the surface on the lens; and (c) graft polymerizing a hydrophilic polymer to the lens surface. According to this invention, the graft polymerization step (c) is facilitated with the use of microwave radiation.

This method utilizes standard plasma oxidation and deposition processes (also referred to as “electrical glow discharge processes”) to provide a thin, durable, hydrophilic surface on the contact lens. With an oxidizing plasma, e.g., O₂ (oxygen gas), water, hydrogen peroxide, air, etc., ammonia and the like, the plasma tends to etch the surface of the lens, creating radicals and oxidized functional groups. When used as the sole surface treatment, such oxidation renders the surface of a lens more hydrophilic; however, the coverage of such surface treatment may be incomplete and the bulk properties of the silicone material remain apparent at the surface of the lens (e.g., silicone molecular chains adjacent the lens surface are capable of rotating, thus exposing hydrophobic groups to the outer surface). Hydrocarbon plasmas, on the other hand, deposit a thin carbon layer (e.g., from a few Angstroms to several thousand Angstroms thick) upon the surface of the lens, thereby creating a barrier between the underlying silicone materials and the outer lens surface. Following the deposition of a carbon layer on the lens to form a barrier, a further plasma oxidation will render the surface more hydrophilic. Thus, the surface of the lens is first subjected to a plasma oxidation, prior to subsequent plasma polymerization to deposit a carbon layer, followed by a final plasma oxidation. The initial plasma oxidation in step (a) prepares the surface of the lens to bind the carbon layer that is subsequently deposited by plasma polymerization on the lens in step (b). This carbon layer or coating provides relatively complete coverage of the underlying silicone material.

Step (c) involves graft polymerizing a hydrophilic polymer to the lens surface so as to render the carbon coating of step (b) hydrophilic. The aforementioned hydrophilic polymers may be employed. In this step, the lens is contacted with a solution containing the hydrophilic polymer, and subjected to microwave radiation.

The following examples illustrate various aspects of the present invention and should not be construed as limiting the invention.

The following examples illustrate various aspects of the present invention and should not be construed as limiting the invention.

### EXAMPLE 1

### Synthesis of Reactive, Hydrophilic Copolymer of N,N-dimethylacrylamide (DMA) and Glycidyl Methacrylate (GMA)—DMA-co-GMA [x=86, y=14]

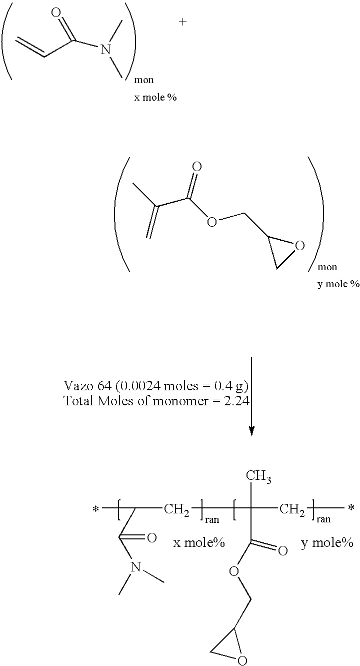

To a 3 L reaction flask were added distilled N,N-dimethylacrylamide (DMA, 192 g, 1.92 moles), distilled glycidyl methacrylate (GMA, 48 g, 0.32 moles) Vazo 64 (AIBN, 0.4 g, 0.0024 moles) and tetrahydrofuran (2000 ml). The reaction vessel was fitted with a mechanical stirrer, condenser, thermal controller and a nitrogen inlet. Nitrogen was bubbled through the solution for 15 minutes to remove any dissolved oxygen. The reaction flask was then heated to 60° C. under a passive blanket of nitrogen for 24 hours. The reaction mixture was then added slowly to 12 L of ethyl ether with good mechanical stirring. The reactive polymer precipitated and was collected by vacuum filtration. The solid was placed in a vacuum oven at 30° C. overnight to remove the ether leaving 213.85 g of reactive polymer (89% yield). The reactive polymer was placed in a desiccator for storage until use.

This general procedure is followed to prepare the reactive polymers listed in the table below.

| DMA | | DMA | GMA | | GMA | AIBN | | volume | Time | Yield |
|---|---|---|---|---|---|---|---|---|---|---|
| grams | moles | x mole % | grams | moles | y mole % | moles | Solvent | ml | (hours) | grams |
| 57 | 0.58 | 97 | 3 | 0.02 | 3 | 0.0006 | toluene | 600 | 20 | 50.4 |
| 54 | 0.54 | 93 | 6 | 0.042 | 7 | 0.0006 | toluene | 600 | 20 | 53.5 |
| 42 | 0.42 | 76 | 18 | 0.13 | 24 | 0.0006 | toluene | 600 | 20 | 46.7 |
| 36 | 0.36 | 68 | 24 | 0.17 | 32 | 0.0006 | toluene | 600 | 20 | 49.8 |

### EXAMPLE 2

### Surface Modification of Poly(HEMA-co-HOHEXMA) Intraocular Lens Implant with Reactive, Hydrophilic Copolymer of N,N-dimethylacrylamide (DMA) and Glycidyl Methacrylate (GMA) From Example 1

This example employs an IOL made from a hydrogel copolymer that is the polymerization product of 2-hydroxyethyl methacrylate (Hema) and 6-hydroxyhexyl methacrylate (Hohexma), i.e., poly(Hema-co-Hohexma).

Poly(Hema-co-Hohexma) IOLs were surface modified or coated by placing the IOLs in a container and adding a 1.0 percent by weight poly(DMA-co-GMA [86/14 mole %]) solution from Example 1 to the container to cover the intraocular lens implants. Then, the container with the IOLs and solution was microwaved for 30 seconds (high setting/Litton Commercial Microwave Magnitron FS-14 EVP) and, in a separate experiment, 60 seconds. The container was then removed from the autoclave and the IOLs were removed from the solution. The intraocular lens implants were then rinsed three times in a buffered saline solution.

### Example 3

### Surface Analysis

Lenses treated in Example 2were analyzed by x-ray photoelectron spectroscopy (XPS) to determine the extent of the applied coating. Results are summarized in the table below. Compared to controls, the coated lens implants contained a unique elemental tag, nitrogen. The nitrogen content of the control lens implants statistically increased when coated from 1.0 to 5.0 percent indicating the poly(DMA-co-GMA) coating had been applied. The 1.0 percent nitrogen on the control lens is usually biological contamination. The level of nitrogen, 3.0 percent, on the coated lens implants is indicative of an approximate 20-angstrom thick coating.

TABLE 1
XPS Results

| | [C] | [O] | [N] | [Si] |
|---|---|---|---|---|
| Control (Uncoated) | | | | |
| Sample 1 | 76.1 | 22.8 | 1.14 | 0.0 |
| Sample 2 | 78.5 | 20.1 | 1.38 | 0.0 |
| Sample 3 | 74.8 | 23.3 | 1.92 | 0.0 |
| Test-30 seconds | | | | |
| Sample 1 | 73.5 | 23.7 | 2.9 | 0.0 |
| Sample 2 | 71.9 | 25.1 | 2.9 | 0.0 |
| Sample 3 | 71.2 | 25.8 | 3.1 | 0.9 |
| Test-60 seconds | | | | |
| Sample 1 | 74.6 | 23.2 | 2.1 | 0.0 |
| Sample 2 | 73.6 | 22.5 | 3.9 | 0.0 |
| Sample 3 | 69.6 | 27.2 | 3.2 | 0.0 |

### Example 4

### Surface Modification of Hydrogel Contact Lenses with Hydrophilic Copolymer of Acrylamide and Acrylic Aid

Soflens 66® contact lenses are manufactured by Bausch & Lomb Incorporated (Rochester, N.Y., USA). These lenses are made of a non-silicone hydrogel copolymer that is the reaction product of a monomer mixture composed mainly of Hema and NVP.

The contact lenses were treated with a solution of 0.5% 30/70 poly(acrylamide)-co-(acrylic acid)(P(A)(AA)). The treatment involved immersing the lenses in the P(A)(AA) polymer solution (acidified to pH=3.5) followed by a 30 second microwave (High setting/Litton Commercial Microwave Magnitron FS-14 EVP). The lenses were washed three time with distilled water and immersed in borate buffer saline. The treated lenses were clear and lubricious.

### Example 5

### Surface Modification of Silicone Hydrogel Contact Lenses with Hydrophilic Copolymer of Acrylamide and Acrylic Acid

PureVision® contact lenses are manufactured by Bausch & Lomb Incorporated. These lenses are made of a silicone hydrogel copolymer that is the reaction product of a monomer mixture composed mainly of NVP, 3-[tris(tri-methylsiloxy)silyl]propyl vinyl carbamate, and a monomeric material of Formula (III).

The lenses were treated with a solution of 0.5% 30/70 poly(acrylarnide)-co-(acrylic acid)(P(A)(AA)). The treatment involved immersing the lenses in the P(A)(AA) polymer solution (acidified to pH=3.5) followed by a 30 second microwave (High setting/Litton Commercial Microwave Magnitron FS-14 EVP). The lenses were washed three time with distilled water and immersed in borate buffer saline. The treated lenses were clear and lubricious.

Many other modifications and variations of the present invention are possible in light of the teachings herein. It is therefore understood that, within the scope of the claims, the present invention can be practiced other than as herein specifically described.

1. A method of modifying the surface of a medical device, comprising:
contacting a surface of the medical device with a solution containing a surface modifying agent; and
subjecting the device surface and surface modifying agent to microwave radiation while the surface modifying agent is in contact with the device surface.

2. The method of claim 1, wherein the surface modifying agent is attached to the device surface by covalent bonding.

3. The method of claim 2, wherein a covalent bond is formed between a reactive group on the surface modifying agent and a complementary reactive group on or near the surface of the device.

4. The method of claim 1, wherein the surface modifying agent is attached to the device surface by ionic bonding.

5. The method of claim 1, wherein the surface modifying agent comprises a proton-donating wetting agent.

6. The method of claim 1, wherein the wetting agent comprises an acrylic acid polymer.

7. The method of claim 1, wherein the surface modifying agent comprises a reactive hydrophilic copolymer.

8. The method of claim 7, wherein the reactive hydrophilic copolymer is a copolymer of a hydrophilic monomer and a monomer containing a reactive group.

9. The method of claim 8, wherein the monomer containing a reactive group contains an epoxy group.

10. The method of claim 1, wherein the device is an ophthalmic lens.

11. The method of claim 1, wherein the device is a contact lens.

12. The method of claim 1, wherein the device is an intraocular lens implant.

13. The method of claim 1, wherein the device is a contact lens.

14. The method of claim 1, wherein the device is formed of a hydrogel copolymer.

15. The method of claim 14, wherein the device is formed of a silicone hydrogel copolymer.

16. The method of claim 1, wherein the device is formed of a silicone material.

17. The method of claim 1, wherein the device surface and surface modifying agent are not subjected to plasma treatment while subjected to microwave radiation.

18. The method of claim 1, comprising:
contacting a surface of a hydrogel contact lens with a solution containing an acrylic acid polymer; and
subjecting the lens surface and acrylic acid polymer to microwave radiation while the acrylic acid polymer is in contact with the lens surface.

19. The method of claim 18, wherein the acrylic acid polymer comprises a copolymer of acrylamide and acrylic acid.

20. The method of claim 1, comprising:
contacting a surface of an intraocular lens with a solution containing a hydrophilic copolymer, said hydrophilic copolymer containing a group reactive with the intraocular lens; and
subjecting the lens surface and the hydrophilic copolymer to microwave radiation while the hydrophilic copolymer is in contact with the lens surface.

21. The method of claim 20, wherein the intraocular lens is formed of a hydrogel polymer that is the polymerization product of a monomer mixture comprising 2-hydroxyethyl methacrylate and 6-hydroxyhexayl methacrylate, and the hydrophilic copolymer is the polymerization product of a monomer mixture comprising N,N-dimethylacrylamide and glycidyl methacrylate.
